(19) United States
(12) Patent Application Publication
(10) Pub. No.: US 2023/0382838 A1
(43) Pub. Date: Nov. 30, 2023

(54) PROCESS FOR THE PREPARATION OF CANNABIDIOL

(71) Applicant: Consort Medical Limited, King's Lynn (GB)

(72) Inventors: Urvish Pandya, Cramling (GB); Sam Cattell, Cramling (GB); Carolina Malago, Cramling (GB); Keren Schultz, Yavne (IL); Alexander Aizikovich, Yavne (IL); Keti Lerman, Yavne (IL)

(21) Appl. No.: 18/032,334

(22) PCT Filed: Oct. 19, 2021
(86) PCT No.: PCT/GB2021/052691

§ 371 Date: Apr. 17, 2023

(30) Foreign Application Priority Data
Oct. 19, 2020 (GB) 2016536.1

(51) Int. Cl. C07C 39/23

(52) U.S. Cl. CPC C07C 39/23

(57) ABSTRACT

There is described a process for the preparation of Cannabidiol (CBD) and derivatives thereof of formula I: (I) in which R¹ is H or —COOR²; 10 wherein R² is C₁₋₆alkyl or aryl; said process comprising contacting a solution of (+)-p-mentha-diene-3-ol, or an ester thereof, with a compound of formula II: (II) in which R¹ and R² are each as herein defined; and an ion-exchange resin as a catalyst.

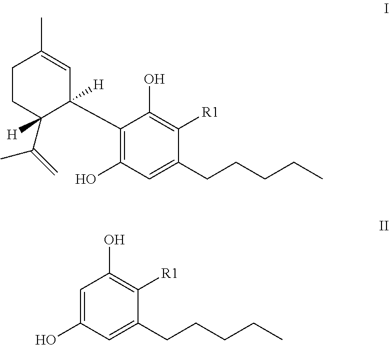

### FIELD OF THE INVENTION

The present invention relates to a process for the synthesis of Cannabidiol and other related cannabinoids namely the methyl ester of Cannabidiolic Acid.

### BACKGROUND OF THE INVENTION

Cannabidiol (CBD) was discovered in 1940. It and accounts for up to 40% of the extract from cannabis plants; and is the major non-psychotropic phytocannabinoid in most cannabis preparations. Cannabidiol has been found to have antiepileptic, anti-anxiety and antidystonia properties in man.

Alongside Cannabidiol many other related natural and synthetic cannabinoids such as Cannabidiolic Acid (CBDa) and the related analogue Cannabidiolic Acid Methyl Ester (CBDa-Me) are also of interest for their potential anti-cancer and anti-nausea properties in man.

Cannabidiol is 2-[1R-3-methyl-6R-(1-methylethenyl)-2-cyclohexen-1-yl]-5-pentyl-1,3-benzenediol or 2-[(6R)-3-methyl-6-prop-1-en-2-ylcyclohex-2-en-1-yl]-5-pentylbenzene-1,3-diol (IUPAC):

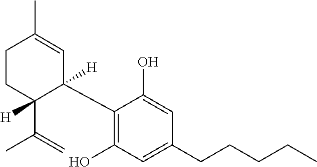 [SMILES: C=C(C)[C@@H]1CCC(C)=C[C@H]1c1c(O)cc(CCCCC)cc1O]

Cannabidiol (CBD) is normally taken to refer to the naturally occurring (−)-enantiomer.

Cannabidiolic Acid Methyl Ester is 2,4-dihydroxy-3-[(1R)-3-methyl-(6R)-(1-methylethenyl)-2-cyclohexen-1-yl]-6-pentyl-benzoic acid methyl ester:

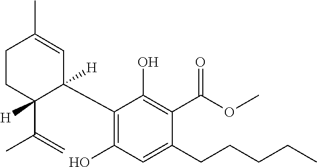 [SMILES: C=C(C)[C@@H]1CCC(C)=C[C@H]1c1c(O)cc(CCCCC)c(C(=O)OC)c1O]

The methyl ester of Cannabidiolic Acid (CBD-Me) is normally taken to refer to the (−)-enantiomer.

Various synthetic routes are available for the production of CBD, but the most efficient route is the condensation of (+)-p-mentha-diene-3-ol with olivetol in the presence of strong acids such as trifluoroacetic acid, p-toluenesulfonic acid, hydrochloric acid, BF₃-etherate (BF₃-Et₂O) or weak acids, such as oxalic, picric or maleic acid. However, such approaches lead to the formation of considerable amounts of two undesired products, the unnatural CBD isomer (abnormal-CBD) and the psychotropic phytocannabinoid tetrahydrocannabinol (THC). Similar conditions to these are used to synthesise CBDa-Me and associated esters.

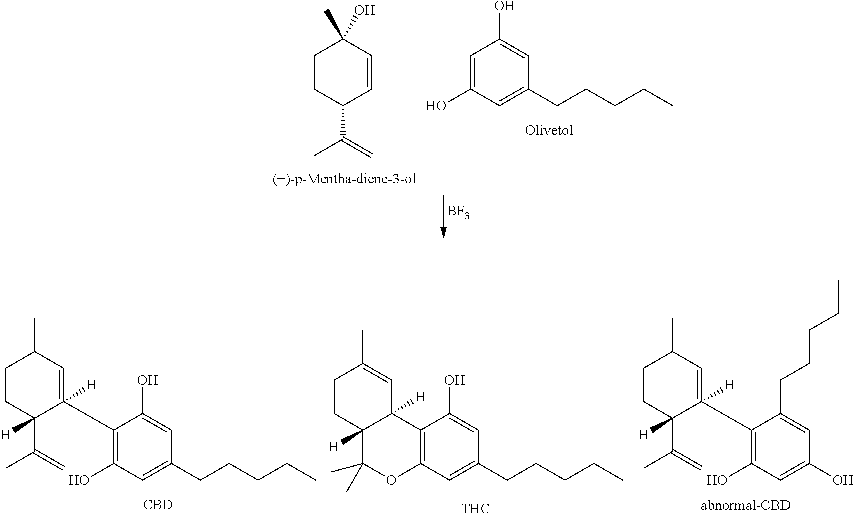

THC is a controlled substance in many territories and has been associated with acute psychosis; therefore, the formation of amounts of THC make CBD production by chemical synthesis complicated. Furthermore, the available synthetic routes only provide low yields of CBD, e.g. around 20%.

International Patent application No. WO 2020/099283 describes a continuous flow synthesis of cannabidiol using a non-supported Lewis acid. However, yields of CBD are still relatively low for a commercially feasible manufacturing method at 30-50%.

Therefore, in view of the low yield of CBD and the formation of undesirable THC in prior art processes, there is a long felt need for a suitable commercial method that would provide an efficient and commercially feasible method of manufacturing CBD.

### SUMMARY OF THE INVENTION

Therefore, an object of the present invention is to provide a novel commercially suitable method of manufacturing CBD.

We have now surprisingly found out that reaction of (+)-p-mentha-diene-3-ol with olivetol:

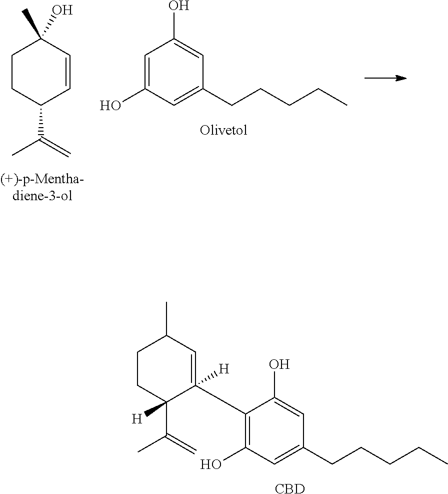

may be carried out in the presence of an ion-exchange resin as catalyst to provide a suitable yield of CBD.

Additionally the reaction of (+)-p-mentha-diene-3-ol with methyl-2,4-dihydroxy-6-pentylbenzoate may also be carried out in the presence of an ion-exchange resin as catalyst to provide a suitable yield of CBDa-Me:

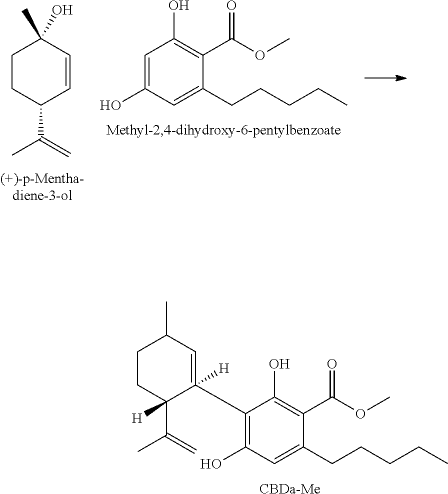

Thus, according to a first aspect of the present invention there is provided a process for the preparation of Cannabidiol (CBD) and derivatives thereof of formula I:

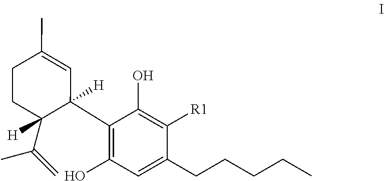

in which R¹ is H or —COOR²;
wherein R² is C₁₋₆alkyl or aryl;
said process comprising contacting a solution of (+)-p-mentha-diene-3-ol, or an ester thereof, with a compound of formula II.

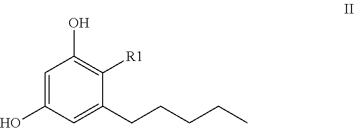

in which R¹ and R² are each as herein defined;
and an ion-exchange resin as a catalyst.

In one aspect of the invention the compound of formula II is olivetol, in which case the compound of formula I prepared by the process of the invention is Cannabidiol (CBD).

In another aspect of the invention the compound of formula II is C₁₋₆alkyl-2,4-dihydroxy-6-pentylbenzoate, in which case the compound of formula I prepared by the process of the invention is the methyl ester of Cannabidiol Acid (CBDa-C₁₋₆alkyl), such as (CBDa-Me).

In yet another aspect of the invention the compound of formula II is an aromatic ester of 2,4-dihydroxy-6-pentylbenzoate, in which case the compound of formula I prepared by the process of the invention is the aromatic ester of Cannabidiol Acid (CBDa-Ar). The term “aromatic ester” shall especially include the phenyl ester and the benzyl ester.

It is well understood that an ion-exchange resin comprises a resin or polymer that acts as a medium for ion-exchange. An ion-exchange resin comprises an insoluble matrix or support structure normally in the form of small microbeads. Generally, the microbeads will comprise an organic polymer substrate. The beads may typically be porous, providing a large surface area for ion-exchange to occur.

Ion-exchange resins can usually be categorised into four main groups:
strongly acidic, typically featuring sulfonic acid groups, or the corresponding salts, such as sodium polystyrene sulfonate or polyAMPS;
strongly basic, typically featuring quaternary amino groups, such as, trimethylammonium groups, e.g. polyAPTAC;
weakly acidic, typically featuring carboxylic acid groups; and
weakly basic, typically featuring primary, secondary, and/or tertiary amino groups, e.g. polyethylene amine.

In a particular aspect of the present invention the ion-exchange resin will be an acidic ion-exchange resin, preferably a strongly acidic ion-exchange resin.

The acidic cation-exchange resin will generally comprise a strongly acidic resin having a sulfonic acid type ion-exchange groups. As such, commercially available acidic cation-exchange resins may suitably be used. Suitable strongly acidic ion-exchange resins which may be mentioned include, but shall not be limited to, sulfonic acid resins, such as, Amberlite®, Amberlyst®, Amberjet®, Diaion®, such as, Diaion SK104H, Diaion SK1B, Diaion SK110 and Diaion SK112; Dowex® (also known as AmberChrom®), such as, Dowex 50WX2, Dowex 50WX4 and Dowex 50WX8; and combinations thereof.

Strongly acidic ion-exchange resins are available in hydrogen form or salt for, e.g. sodium form. In the process of the present invention the ion-exchange resin is preferably in hydrogen form.

The acidic cation-exchange resin for use in the process of the invention is not particularly limited, but a porous resin is preferred. When a porous resin is used, it is desirably a macroporous resin; that is, having pores that are from about 20 to about 100 nm in diameter.

Esters of (+)-p-mentha-diene-3-ol are well known in the art, or an ester thereof. Examples of such esters include, but shall not be limited to, formyl, —OC(O)C₁₋₄alkyl such as acetyl (Ac or —C(O)CH₃), methoxyacetyl, chloroacetyl, dichloroacetyl, trichloroacety, trifluoroacetyl, triphenylmethoxyacetyl, phenoxyacetyl, benzoylformyl, benzoyl (Bz or —C(O)C₆H₅), benzyloxycarbonyl (Cbz or —C(O)—O—CH₂C₆H₅), methoxycarbonyl, tert-butoxycarbonyl, isopropoxycarbonyl, diphenylmethoxycarbonyl or 2-(trimethylsilyl)ethoxycarbonyl and the like. Examples of groups include: alkyl silyl groups such as trimethylsilyl (TMS), tert-butyldimethylsilyl, triethylsilyl, triisopropylsilyl and the like. In a preferred aspect of the invention the (+)-p-mentha-diene-3-ol is in free form.

C₁₋₆alkyl esters of 2,4-dihydroxy-6-pentylbenzoate are well known in the art. Examples of such esters include, but shall not be limited to, linear alkyl esters such as methyl esters, ethyl esters, propyl esters etc., branched alkyl esters such as tert-butyl ester, isopropyl esters etc. A preferred C₁₋₆alkyl ester of 2,4-dihydroxy-6-pentylbenzoate is the methyl ester. Aromatic esters of 2,4-dihydroxy-6-pentylbenzoate include, but shall not be limited to, phenyl esters and benzylic esters.

In the process of the invention the ratio of (+)-p-mentha-diene-3-ol, or an ester thereof, to the compound of formula II, e.g. olivetol, methyl-2,4-dihydroxy-6-pentylbenzoate or an alternate ester thereof, may vary. The ratio of (+)-p-mentha-diene-3-ol, or an ester thereof, to the compound of formula II may be from about 1:1 to about 1:2 molar ratio, preferably a 1:1 molar ratio.

The reaction may be carried out in a suitable organic solvent, a mixture of organic solvents or an organic solvent and water. Such solvents include, but shall not be limited to, C₁-C₃ chlorinated solvents, such as, dichloromethane; or an ethereal solvent, such as, methyl-tertbutyl ether, tetrahydrofuran, 1,4-dioxane or 2-methyltetrahydrofuran; or alkyl esters, such as, ethyl acetate; or a hydrocarbon solvent, such as, heptane or cyclohexane; or an aromatic solvent, such as, toluene; or an alcohol solvent, such as, ethanol or isopropanol. The preferred solvents are cyclohexane or dichloromethane.

In the process of the invention the concentration of the (+)-p-mentha-diene-3-ol, or an ester thereof, and the compound of formula II, e.g. olivetol, methyl-2,4-dihydroxy-6-pentylbenzoate or an alternate ester thereof, may vary. The concentration of each solute, which may be the same or different, may be from about 0.01M to about 0.5M.

The reaction temperature varies and is from about −20° C. to about 80° C.; and is preferably about 20° C.-25° C.

The reaction successfully runs in a batch process utilising standard wet chemistry techniques with typical reaction times ranging from 3 to 48 hours; the reaction time is dependent on which variables are selected from those outlined above. The loading for the strong acid H form resin ranges from 1-200% w/w w.r.t. the compound of formula II, e.g. olivetol, methyl-2,4-dihydroxy-6-pentylbenzoate or an alternate ester thereof, with the optimal loading primarily dependent on resin type and solvent.

However, the reaction may also be adapted to a run as a continuous process wherein a strong acid ion exchange resin in its H form is packed into a column which has the facility to be heated or cooled. The reaction mixture, either as one stream or two separate streams (i.e. one stream containing (+)-p-mentha-diene-3-ol, or an ester thereof, and one containing the compound of formula II, e.g. olivetol, methyl-2,4-dihydroxy-6-pentylbenzoate or an alternate ester thereof, is pumped through the column and then recirculated as required to achieve suitable conversion.

The residence time of reaction mixture with the ion-exchange resin may vary and may be from about 1 to about 60 minutes, for example, from about 1 to about 30 minutes, or from about 10 to about 30 minutes. When an ester of (+)-p-mentha-diene-3-ol or an alternate ester of 2,4-dihydroxy-6-pentylbenzoate is used, the residence time of the reaction mixture with the ion-exchange resin may vary.

According to a further aspect of the invention the process provides cannabidiol (CBD), and derivatives thereof, having a substantially limited, i.e. low, amount of THC.

Thus, according to a further aspect of the invention there is provided Cannabidiol (CBD) and derivatives thereof of formula I.

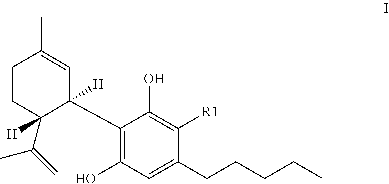

in which R¹ is H or —COOR²;
wherein R² is C₁₋₆alkyl or aryl;
prepared by the process as herein described.

Cannabidiol (CBD) and derivatives thereof of formula I as herein described are advantageous in that, inter alia, they exhibit respectable yields and negligible levels of tetrahydrocannabinol (THC).

Thus, according to a yet further aspect of the invention there is provided Cannabidiol (CBD) and derivatives thereof of formula I as herein described, having substantially limited, i.e. low, amounts of THC.

According to a further aspect of the invention there is provided a pharmaceutical composition comprising Cannabidiol (CBD) and derivatives thereof of formula I.

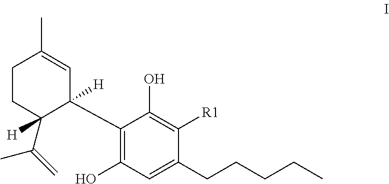

in which R¹ is H or —COOR²;
wherein R² is C₁₋₆alkyl or aryl;
prepared according to the process herein described, said pharmaceutical composition will typically include Cannabidiol (CBD) or derivatives thereof in association with a pharmaceutically acceptable adjuvant, diluent or carrier.

The invention will now be described by way of example only. These examples use Olivetol and (+)-p-mentha-diene-3-ol but this procedure is equally applicable to esters of 2,4-dihydroxy-6-pentylbenzoic acid and (+)-p-mentha-diene-3-ol.

### BATCH EXAMPLE

Olivetol (1.0 g) and (+)-p-mentha-diene-3-ol (0.9 mL) are charged to a round bottomed flask equipped with a stirrer. Cyclohexane (30 mL) is added and the mixture is stirred at 25° C. until all solids dissolve, at this point Dowex 50WX2 H-form 50-100 mesh (1.0 g) is added. The reaction is monitored by HPLC and is typically complete after 4 hours. Once complete the mixture is filtered and then concentrated under reduced pressure. The resulting residue is purified by column chromatography to give Cannabidiol (Typical isolated yield 35%-40%).

### CONTINUOUS SINGULAR FEEDSTOCK EXAMPLE

Dowex 50WX2 H-form 50-100 mesh (10.7 g) is packed into a jacketed column, the column is preheated to 25° C. This packed column is then flushed with cyclohexane (10 mL). A solution of Olivetol (1.0 g, mmol) and (+)-p-mentha-diene-3-ol (0.9 mL, mmol) in cyclohexane (25 mL) is prepared, this is then pumped through the column. The flow rate is adjusted to provide a residence time of 10 minutes. The reaction mixture is recirculated until off line HPLC analysis confirms that the reaction is complete, this is typically 1 hour. Once the reaction is complete recirculation is stopped and the column is flushed with cyclohexane (10 mL). The reaction mixture is concentrated under reduced pressure and then purified by column chromatography to give Cannabidiol (Typical isolated yield (40-45%).

1: A process for the preparation of Cannabidiol (CBD) and derivatives thereof of formula I: 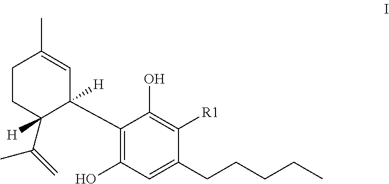
in which R¹ is H or —COOR²;
wherein R² is C₁₋₆alkyl or aryl;
said process comprising contacting a solution of (+)-p-mentha-diene-3-ol, or an ester thereof, with a compound of formula II: 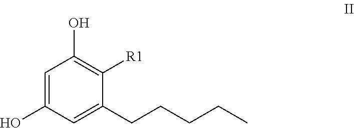
in which R¹ and R² are each as herein defined;
and an ion-exchange resin as a catalyst.

2: A process according to claim 1 wherein the compound of formula II is olivetol, in which case the compound of formula I is Cannabidiol (CBD).

3: A process according to claim 1 wherein the compound of formula II is C₁₋₆alkyl-2,4-dihydroxy-6-pentylbenzoate, in which case the compound of formula I prepared by the process of the invention is the methyl ester of Cannabidiol Acid (CBDa-C₁₋₆alkyl).

4: A process according to claim 3 wherein the C₁₋₆alkyl esters of 2,4-dihydroxy-6-pentylbenzoate include esters such as methyl esters, ethyl esters, propyl esters etc., branched alkyl esters such as tert-butyl ester, isopropyl esters etc., and optionally wherein the C₁₋₆alkyl ester of 2,4-dihydroxy-6-pentylbenzoate is the methyl ester (CBDa-Me).

5. (canceled)

6: A process according to claim 1 wherein the compound of formula II is an aromatic ester of 2,4-dihydroxy-6-pentylbenzoate, in which case the compound of formula I is the aromatic ester of Cannabidiol Acid (CBDa-Ar), and optionally wherein the aromatic esters of 2,4-dihydroxy-6-pentylbenzoate include, but shall not be limited to, phenyl esters and benzylic esters.

7. (canceled)

8: A process according to claim 1 wherein the ion-exchange resin comprises an acidic ion-exchange resin.

9: A process according to claim 8 wherein the ion-exchange resin comprises a strongly acidic ion-exchange resin.

10: A process according to claim 9 wherein the ion-exchange resin comprises sulfonic acid type ion-exchange groups.

11: A process according to claim 1 wherein the ion-exchange resin comprises a porous resin, and optionally wherein the ion-exchange resin comprises a porous resin having pores of from about 20 to about 100 nm in diameter.

12. (canceled)

13: A process according to claim 1 wherein the ion-exchange resin is selected from the group consisting of Amberlite®, Amberlyst®, Amberjet®, Diaion®, such as, Diaion SK104H, Diaion SK1B, Diaion SK110 and Diaion SK112; Dowex® (also known as AmberChrom®), such as, Dowex 50WX2, Dowex 50WX4 and Dowex 50WX8; and combinations thereof.

14: A process according to claim 1 wherein the ion-exchange resin is in hydrogen form.

15: A process according to claim 1 wherein the (+)-p-mentha-diene-3-ol is in free form.

16: A process according to claim 1 wherein the ratio of (+)-p-mentha-diene-3-ol, or an ester thereof, to the compound of formula II, is from about 1:1 to about 1:2 molar ratio.

17: A process according to claim 1 wherein reaction is carried out in a suitable organic solvent or a mixture of an organic solvent and water.

18: A process according to claim 17 wherein the suitable organic solvent includes C₁-C₃ chlorinated solvents, such as, dichloromethane; or an ethereal solvent, such as, methyl-tertbutyl ether, tetrahydrofuran, 1,4-dioxane or 2-methyltetrahydrofuran; or alkyl esters, such as, ethyl acetate; or a hydrocarbon solvent, such as, heptane or cyclohexane; or an aromatic solvent, such as, toluene; or an alcohol solvent, such as, ethanol or isopropanol; or any combination thereof, and optionally wherein the suitable organic solvent cyclohexane or dichloromethane or a combination thereof.

19. (canceled)

20: A process according to claim 1 wherein the concentration of each solute is from about 0.01M to about 0.5M.

21: A process according to claim 1 wherein the reaction temperature is from about −20° C. to about 80° C., and optionally wherein the reaction temperature is from about 20° C. to about 25° C.

22. (canceled)

23: A process according to claim 1 wherein the residence time of reaction mixture with the ion-exchange resin is from about 1 to about 60 minutes.

24. (canceled)

25. (canceled)

26: Cannabidiol (CBD) and derivatives thereof of formula I: 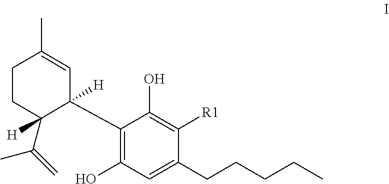
in which R¹ is H or —COOR²;
wherein R² is C₁₋₆alkyl or aryl;
prepared by the process according to claim 1.

27. (canceled)

28: A pharmaceutical composition comprising Cannabidiol (CBD) and derivatives thereof of formula I: 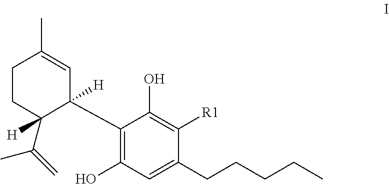
in which R¹ is H or —COOR²;
wherein R² is C₁₋₆alkyl or aryl;
wherein said pharmaceutical composition includes Cannabidiol (CBD) or derivatives thereof of formula I: in association with a pharmaceutically acceptable adjuvant, diluent or carrier.

29. (canceled)
